(19) United States
(12) Patent Application Publication
(10) Pub. No.: US 2013/0182217 A1
(43) Pub. Date: Jul. 18, 2013

(54) FUNDUS CAMERA

(71) Applicant: Optovue, Inc., Fremont, CA (US)

(72) Inventors: Yeou-Yen Cheng, Saratoga, CA (US); Jay Wei, Fremont, CA (US)

(73) Assignee: Optovue, Inc., Fremont, CA (US)

(21) Appl. No.: 13/678,488

(22) Filed: Nov. 15, 2012

Related U.S. Application Data
(60) Provisional application No. 61/561,266, filed on Nov. 18, 2011.

(51) Int. Cl. A61B 3/12

(52) U.S. Cl. CPC A61B 3/12
USPC 351/206

(57) ABSTRACT

An ophthalmic imaging apparatus is provided. The apparatus includes a fundus illumination system, the fundus illumination system includes a spatially interlaced light source array of one or more wavelength bands and a focus index illumination light source where the focus index illumination light source is mounted on a non-moving part of the ophthalmic imaging apparatus, a focus index optical assembly, and a fundus imaging system.

### RELATED APPLICATIONS

This application claims priority to U.S. Provisional Application 61/561,266, filed on Nov. 18, 2011, which is herein incorporated by reference in its entirety.

### BACKGROUND

1. Field of Invention

Embodiments of the invention relate generally to an ophthalmic photographing apparatus.

2. Description of Related Art

In a conventional fundus camera, a focus index, such as a split-bar pattern, is generated from a focus index projection system using a light source with wavelength in the range of dark red or near infrared. The focus index projection is then branched into a fundus illumination path through a beam splitter or a flipping mirror (shutter). Another way of branching the focus index projection into the fundus illumination path is through the projection of the focus index on to a retractable stick minor which is conjugate to the fundus of a subject's eye (Ef). The split-bar pattern is then re-imaged at the fundus (Ef) of the eye under examination after the illumination beam passes through the ocular lens and the eye. The image of the fundus (Ef), usually obtained with Near Infra Red (NIR) for observation or alignment purpose, superimposed with the focus index, can then be captured by a sensor located at the end of the imaging path. The operator then judges the degree of focus by looking at the alignment of the two halves of the split bar image. When the focus setting is correct, the two halves of the split bar image become aligned; otherwise, the two halves are misaligned, depending on the direction and amount of defocus. After the operator adjusted the focus and triggered image acquisition, a control system of the fundus camera turns off the NIR light sources for both the fundus and the focus index illumination and retracts the stick mirror out of the main illumination path before turning on the flash light (white) to capture a color fundus image.

During the focus adjustment of the conventional fundus camera, the entire focus index projection unit, including the light source, mask, condenser lens, bi-prism, slit, folding mirror, projection lens, and the solenoid retractable stick mirror, are moved along the optical axis to synchronize the movement of the focusing lens, which is usually located after an imaging aperture stop, through a mechanical linkage, such as a gear system. This conventional approach requires a large space to accommodate the movement of the entire focus index projection unit, the focusing lens in the imaging path, and the mechanical linkage, and, therefore, is not suitable for a low-cost compact system design.

In an attempt to solve this problem, a simplified focus index projection system was previously disclosed where the slit and the bi-prism were attached to a transparent plate to deflect the light from the fundus illumination light source, the whole assembly can be flipped in-and-out and moved longitudinally during focusing. However, sharing the light source of the fundus illumination with that of the focus index illumination would result in unsatisfactory visibility of the focus index observed by the operator.

Another method was also disclosed to enhance the visibility of the focus index by passing the focus index illumination light through the central opening of a crystalline lens diaphragm. However, the opening hole at the central blocking disk of the crystalline lens diaphragm results in leakage or ghost light.

A method to avoid the leakage from the central opening of the crystalline lens diaphragm was previously disclosed. In this method, the focus index illuminating light source, green Light Emitting Diode (LED), was mounted on the mechanical arm holding the focus index optical assembly. Since the arm and the focus index together need to be flipped in and out at a rapid rate during each switching between the observation mode and the image capturing mode, the light source would inevitably experience shock and vibration, and this method would result in reliability issues. Also, the visible light, such as the green LED disclosed, is not suitable for non-mydriatic application as the patient's pupil size can be sensitive to the visible light generated by the green LED.

Therefore, there is a need for systems and methods to generate focus index of a fundus camera with good visibility, reliability, and enhanced user-friendliness that can be suitable in a compact design.

### SUMMARY

In accordance with some embodiments, an ophthalmic imaging apparatus is provided. An ophthalmic imaging apparatus for capturing images of an eye according to some embodiments includes a fundus illumination system, the fundus illumination system includes a spatially interlaced light source array of one or more wavelength bands and a focus index illumination light source where the focus index illumination light source is mounted on a non-moving part of the ophthalmic imaging apparatus, a focus index optical assembly, and a fundus imaging system.

### BRIEF DESCRIPTION OF THE DRAWINGS

FIG. 1 is a cross sectional view of a compact fundus camera in accordance with some embodiments of the present invention.

FIGS. 2A and 2B show an exemplary crystalline lens diaphragm with small prism mirror attached at a central light blocking disk.

FIGS. 2C and 2D show another exemplary crystalline lens diaphragm with small prism mirror attached at a central light blocking disk.

FIGS. 3A, 3B, 3C, 3D, 3E, and 3F show an example of an interlaced white and NIR ring LED arrays.

FIGS. 4A and 4B show an example of a focus index optical assembly with multiple fixation targets.

FIGS. 5A and 5B show the lens slider mounted with different compensation lenses according to some embodiments of the present invention.

### DETAILED DESCRIPTION

Embodiments of the present invention are described herein with reference to the exemplary drawings. In the drawings, elements having the same element designation have the same or similar functions.

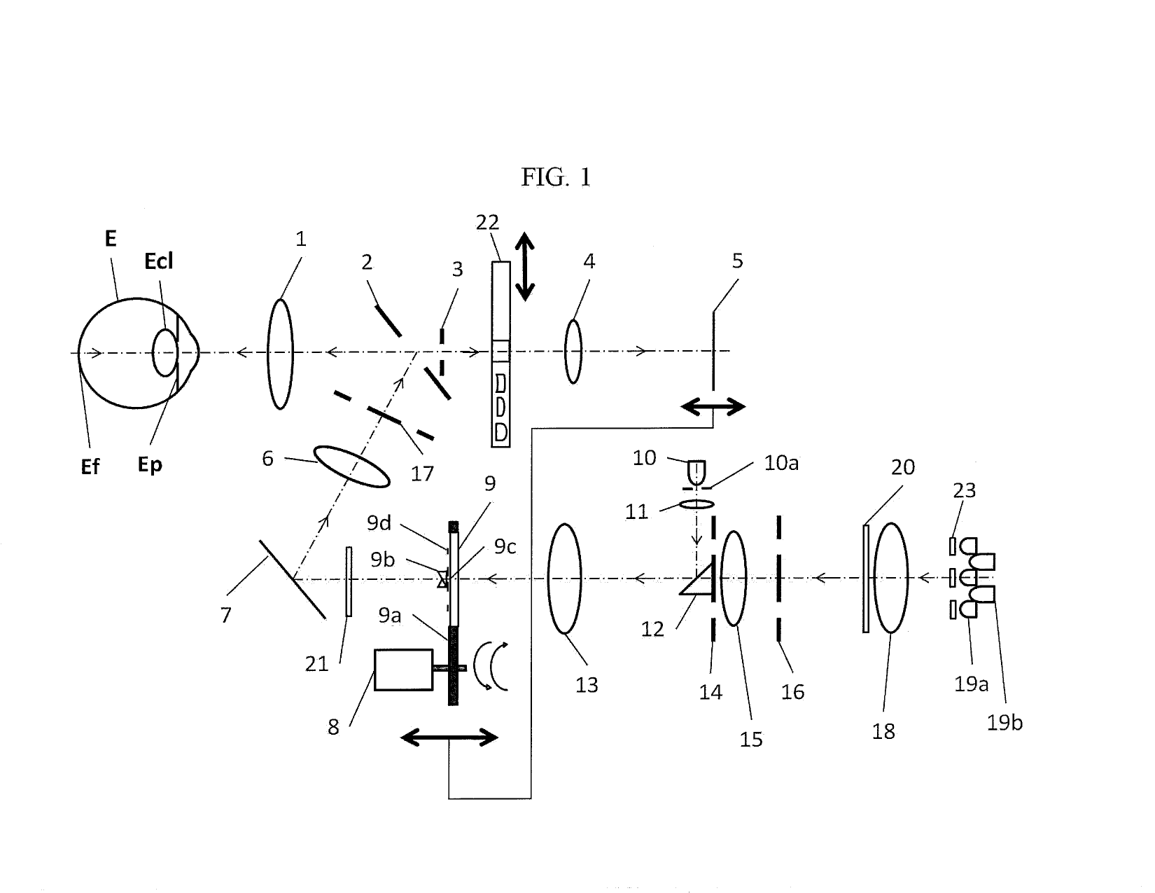

FIG. 1 shows a cross sectional view of a compact fundus camera in accordance with some embodiments of the present invention. As shown in FIG. 1, some embodiments of the fundus camera include an ocular lens 1; hole mirror 2; aperture stop 3; relay lens system 4; a sensor 5; a relay lens 6; a mirror 7; a solenoid 8; an optical assembly 9 with housing structure 9a, bi-prism 9b, focus index 9c, and fixation targets 9d; a focus index illuminating light source 10; a field stop 10a; a relay lens 11; a small folding mirror 12; a second relay lens 13; a crystalline lens diaphragm 14; relay lens 15; a ring aperture plate 16; an aperture 17; a condenser lens 18; LED ring arrays 19a and 19b; a diffuser plate 20; black dot plate 21; a lens slider 22; and filters 23. As shown in FIG. 1, light from light source 10 can be directed by folding mirror 12 through second relay lens 13, optical assembly 9, black dot plate 21, mirror 7, lens 6, aperture 17, hole mirror 2, and ocular lens 1 to the eye. Further, light from LED ring arrays 19a and 19b can be directed through lenses 18, 15, 13, 6, and 1 to the eye. Light from the eye can be directed through lens slider 22 and lens 4 onto sensor 5. Although lenses 1, 4, 6, 13, 15, and 18 shown in the FIG. 1 are all illustrated as a single-element lens, some or all of them can be multi-element lenses. This system is further described below.

The focus index illuminating light source 10, which can be a NIR LED, is mounted on a fixed part. Such fixed part, for example, can be a lens housing mounted on a base structure of the apparatus. In some embodiments, this fixed part is kept further away from the movable focus index optical assembly 9 to minimize the vibration or shock energy by-product generated from the rapid in-and-out retraction motion of the focus index optical assembly 9 during each switching cycle between an observation mode and an image acquisition mode. The reliability of the focus index illumination can be improved by reducing the vibration and shock by-product. Such embodiments have a further advantage of removing the focus index illuminating light source 10 from the focus index optical assembly 9 so that additional space becomes available between the relay lens 13 and the black dot plate 21 for wider range of focus adjustment.

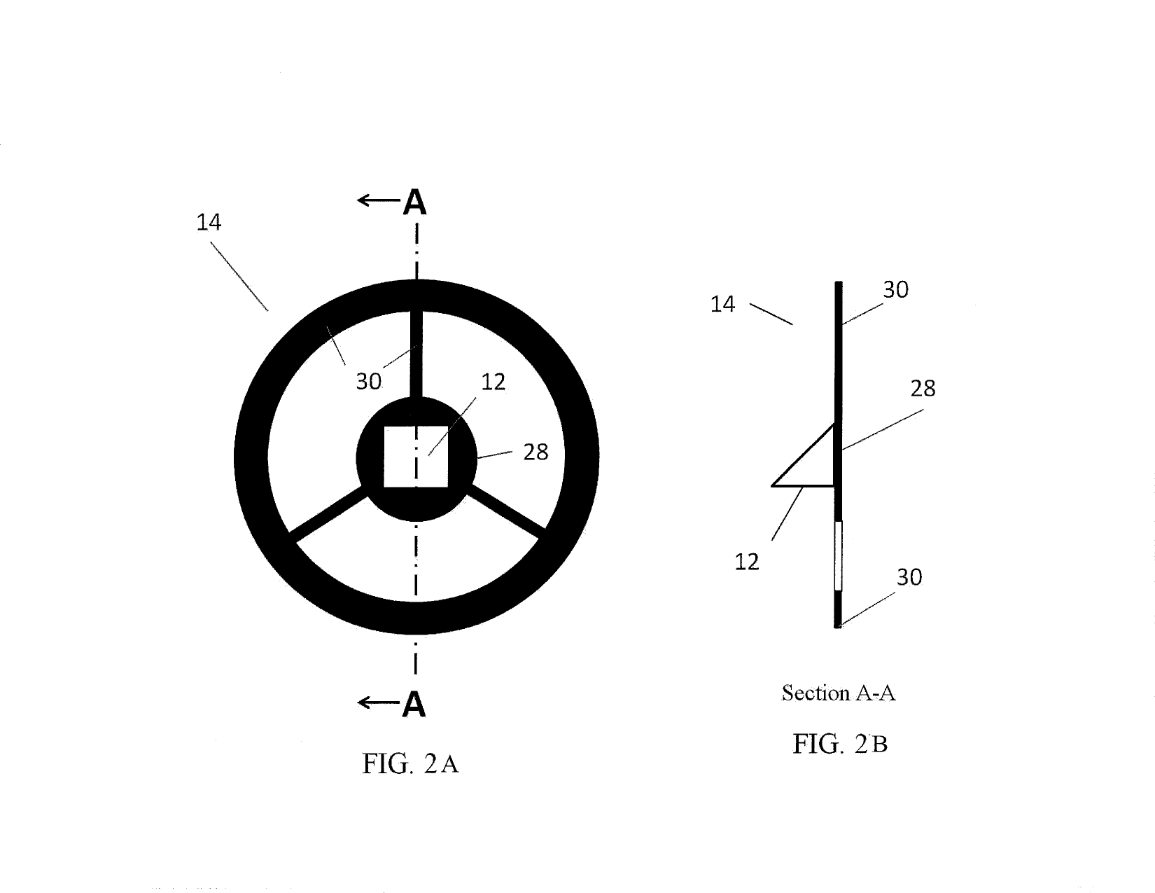

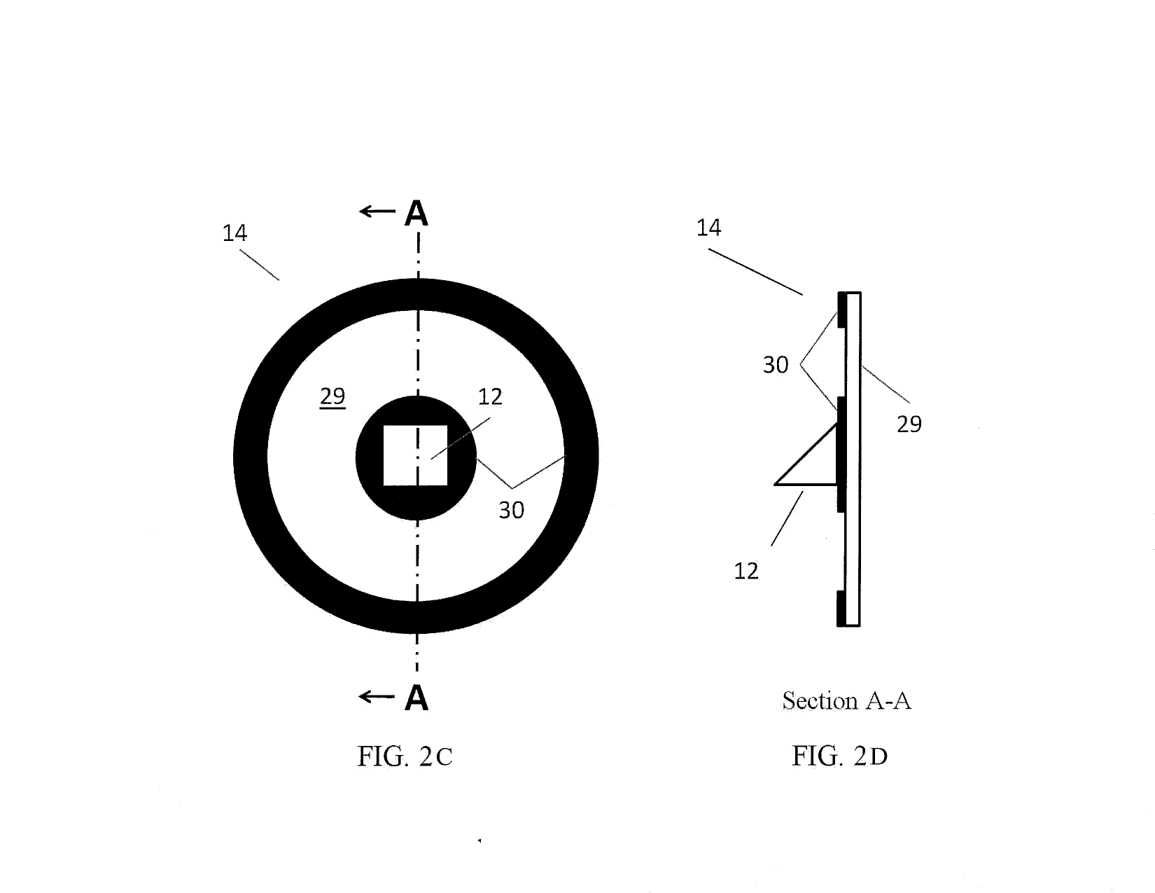

A black dot plate 21 is commonly used in a fundus camera setup to eliminate surface reflection of the ocular lens 1. In some embodiments, a field stop 10a is attached in front of the light source 10, such as a NIR LED, as shown in FIG. 1, so that it is re-imaged by the relay lens 11 to a position near the front focal plane of the second relay lens 13 of the fundus illumination path. The second relay lens 13 re-images the crystalline lens diaphragm 14 to a surface close to the back surface of the crystalline lens (Ecl) of the eye (E), with relay lens 6, and ocular lens 1. In this arrangement, the relay lens 13 also serves as the collimating lens for the focus index illuminating light beam generated from the light source 10. A small folding mirror 12, such as a prism mirror, can be attached onto and hidden from ring arrays 19a and 19b behind the central disk 28 of the crystalline lens diaphragm 14 to minimize interference with the fundus illumination beam when passing through the ring opening of the diaphragm 14. The construction of the prism mirror on the crystalline lens diaphragm 14 is described in details below and is shown in embodiments of diaphragm 14 shown in FIGS. 2A, 2B, 2C, and 2D.

After being reflected by the small folding mirror 12, the optical axis of the focus index illuminating beam coincides with that of the lens 13 and the focus index 9c. In some embodiments, the size of the folding mirror 12 and the position of the combination of the light source 10 and the field stop 10a can be adjusted to minimize stray light from the focus index illumination beam by minimizing the beam size illuminating the central part 44 of the focus index optical assembly 9.

FIGS. 2A, 2B, 2C, and 2D show examples of crystalline lens diaphragms 14 in accordance to some embodiments of the present invention. FIGS. 2A and 2B illustrate a diaphragm constructed from material that is capable of light blocking/absorption. As is shown in FIG. 2A, diagram 14 is formed of a central disk 28 with supporting structures 30. FIG. 2B illustrates a cross section along the A-A direction illustrated in FIG. 2A.

FIGS. 2C and 2D show another example diaphragm 14 constructed by depositing a thin layer of light blocking material 30 onto a translucent material 29. FIG. 2D illustrates a cross section along the A-A direction illustrated in FIG. 2C.

Returning to FIG. 1, in some embodiments, in the fundus observation mode, illumination can be achieved by turning on the NIR LED ring array 19b of a dual band interlaced LED ring arrays 19a and 19b and the focus index illuminating light source 10. According to some embodiments, the spatially interlaced dual-band LED ring array can be arranged on a single Printed Circuit board (PCB) or separated into multiple layers with supporting structure holding the two interlaced LED ring arrays 19a and 19b together.

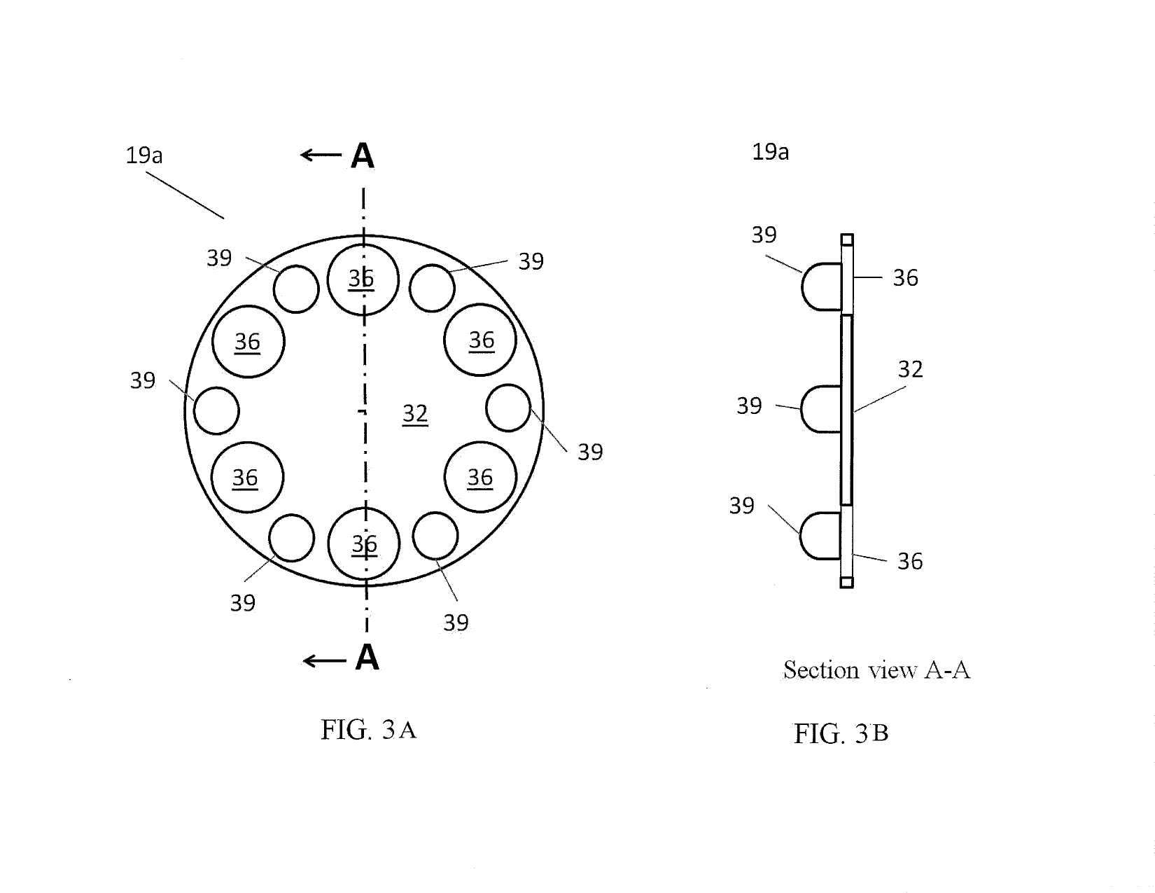

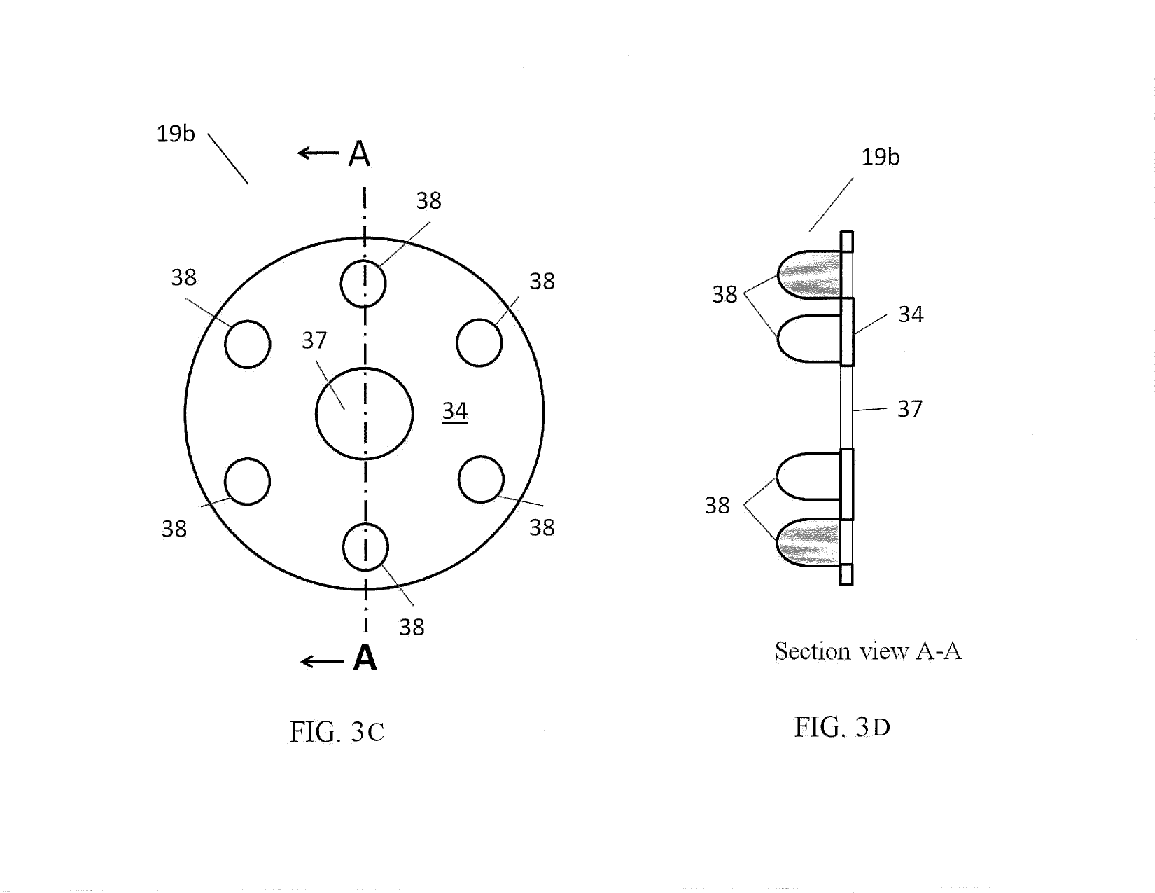

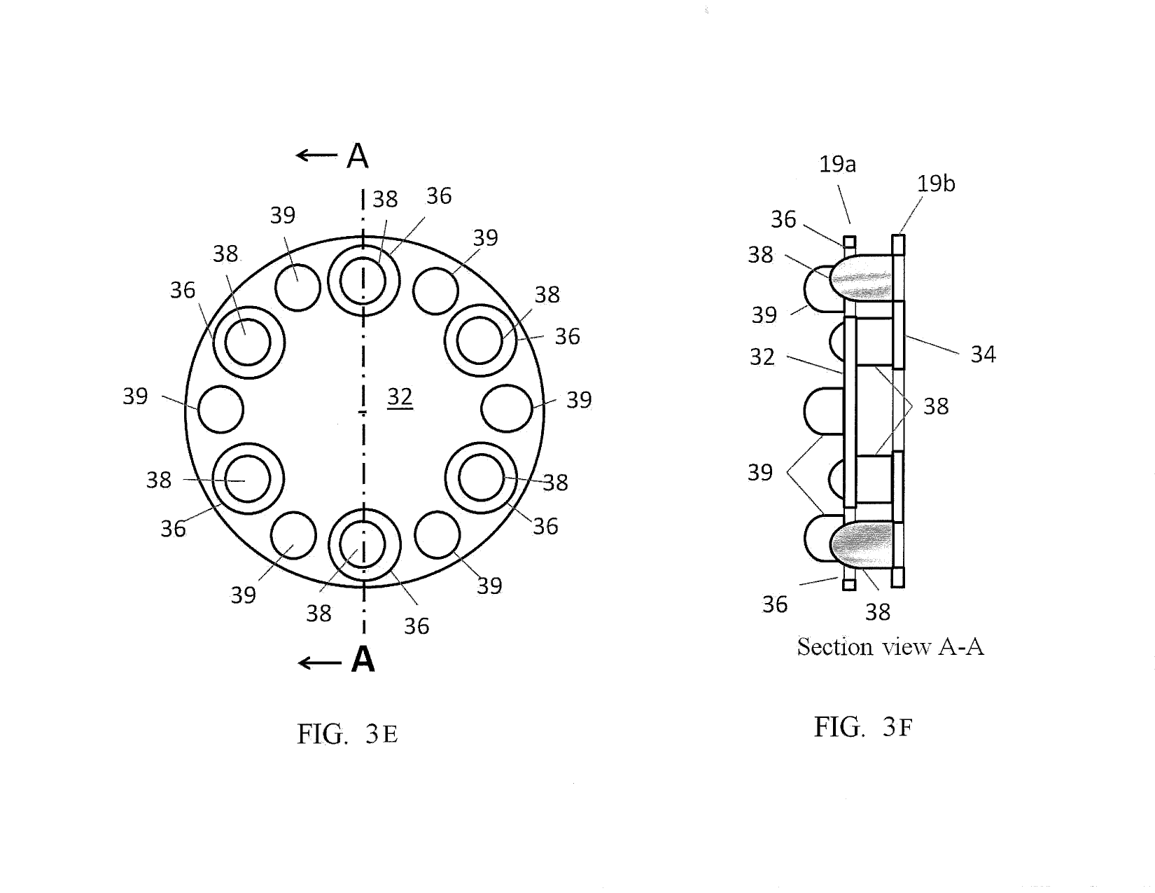

An example of the interlaced ring arrays is shown in FIGS. 3E and 3F. FIGS. 3E and 3F show a ring array which constitutes of two layers of multiple LEDs. As shown in FIG. 3F, LED ring array 19a includes LEDs 39 mounted on printed circuit board 32. LED ring array 19b includes LEDs 38 mounted on printed circuit board 34. LEDs 38 are arranged to insert through holes 36 on printed circuit board 32. As shown in FIG. 3E, then, a ring of LEDs 39 and LEDs 38 are formed. The LEDs 39 can also be arranged in a ring array, evenly spaced apart, and interlaced spatially with LEDs 38 so that each NIR LED can illuminate the condenser lens 18 through the open holes between adjacent white LEDs of the first layer. As can be appreciated by a person of ordinary skill in the art, the order of the LED layers and the combination of the visible band and the NIR band can be alternatively arranged within the spirit of the subject invention.

FIGS. 3A and 3B illustrate LED ring 19a. As shown in FIG. 3A, LEDs 39 are arranged in a ring on a printed circuit board 32. Openings 36 in printed circuit board 32 are interspersed between LEDs 39. FIG. 3B illustrates a cross section along the A-A direction of LED ring 19a.

An example composite of these 2 layers is shown in FIG. 3E. One of the advantages of this approach is the elimination of the need of a dichroic filter to combine the light beams of the different wavelength bands from each of the two separated LED ring arrays 19a and 19b. In some embodiment, the NIR light generated from the array 19b is focused on the ring aperture plate 16 through the opening 36 of a mount, such as the PCB of the white LED arrays 19a as shown in FIG. 3A, a condenser lens 18 and a diffuser plate 20 which makes the illumination more uniform across the fundus (Ef) image plane. The ring aperture plate 16 is conjugate with a position between the pupil (Ep) and the cornea of the eye through the relay lenses 15, 13, and 6, the hole mirror 2, and the ocular lens 1. The crystalline lens diaphragm 14 is conjugate with the back surface of the crystalline lens (Ed) through relay lenses 13 and 6, and the ocular lens 1. Also, in this exemplary optical setup, the cornea ring aperture 17 is conjugate with the cornea.

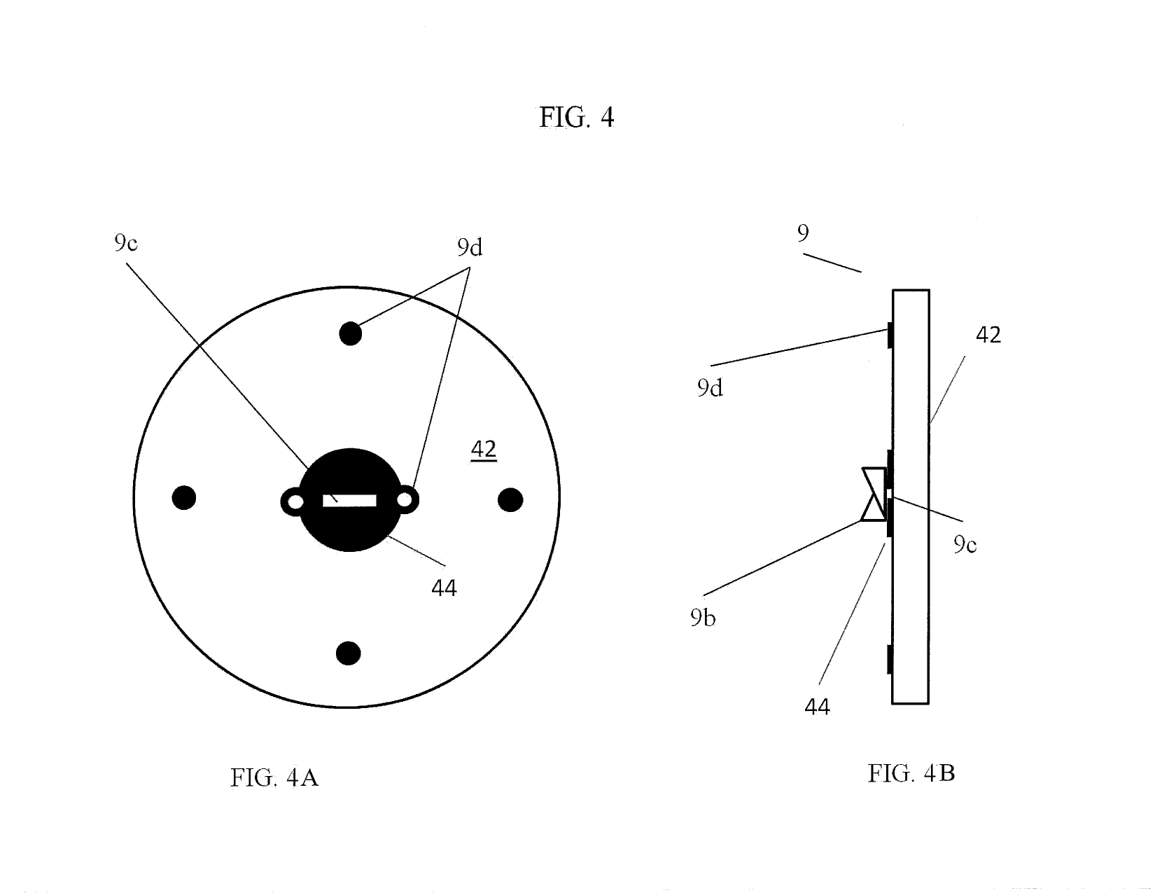

FIGS. 4A and 4B shows an exemplary drawing of a portion of optical assembly 9. FIG. 4A provides a planar view and FIG. 4B provides a cross-sectional view of optical assembly 9. As shown in FIG. 4A, optical assembly 9 includes a covering of thin light-blocking material 44 on a translucent plate 42 to form focus index 9c and fixation targets 9d. Focus index 9c can be a slit opening surrounded by a light-blocking central disk. Multiple fixation targets 9d can be black dots or small openings of any useful shape and size. These fixation targets can be used to stabilize the eye during examination by drawing the patient's attention to any one of these fixation target(s). Note that this method provides passive fixation in a sense that the target illumination is shared with the fundus illumination light source and does not need any additional fixation light source for each fixation position as in the case of the conventional fundus cameras and further save the cost and power of the system. The longitudinal position of these fixation targets 9d relative to that of the focus index 9c can be adjusted to compensate for the field curvature and the index of refraction of the bi-prism 9b so that images of both the fixation targets and the focus index are at focus together at the fundus (Ef). In some embodiments, as shown in FIG. 4B, the bi-prism 9b is attached on top of the focus index 9c to deflect the incident beam into two opposite directions. FIG. 4A is a top view of the exemplary optical assembly 9 with the patterns for the focus index and the fixation targets. FIG. 4B shows the side view of the translucent plate of FIG. 4A showing the bi-prism 9b attached at the top of the focus index 9c. The focus index optics assembly 9 can be held in position by a mechanical housing structure 9a (FIG. 1) fastened on the shaft of the solenoid 8 so that the focus index optics assembly 9 can be flipped in-and-out of the fundus illumination path when the operator switches between the observation mode and the image capturing mode.

As shown in FIG. 1, when the operator adjusts the focus of the fundus camera system, the combination of the focus index optical assembly 9 and the solenoid 8 mounted on a translation stage (not shown) can be moved longitudinally together with the movement of the sensor 5. The movement can be at different rates facilitated by a CAM wheels structure, a gear system or other commonly used methods to control mechanical movement. These embodiments described in FIG. 1 eliminate the need for a focusing lens since the sensor 5 can be used as part of the focus adjustment.

Since the focus index 9c is conjugate with the fundus (Ef), the split-bar pattern is superimposed onto the fundus image captured by the sensor 5 through the ocular lens 1, the central opening of the hole minor 2, the aperture stop 3, the lens slider 22, and the relay lens system 4. The split-bar pattern can then be displayed on a display device so that the operator can observe and adjust for focusing.

The sensor 5 in some embodiments is a dual-band sensor which can capture both color and NIR images. An example of this type of sensor can be constructed by removing the IR cut filter of a typical solid-state sensor, such as a color CMOS or a CCD sensor; where the silicon material is sensitive to visible wavelength band and NIR wavelength band up to around 1,000 nm. This approach has the advantage of using only one sensor for both the observation mode (using NIR light) and the image capturing mode (using visible light). Removing the IR cut filter has the advantage of allowing the sensor to capture the dark red spectrum of the white LED illumination which penetrates deeper into the choroid area of the eye; on the other hand, it can blur the color image slightly due to chromatic aberration. In some embodiments, this disadvantage is overcome here by attaching small IR cut filters 23 used for typical cell phone cameras in front of each white LED 19a.

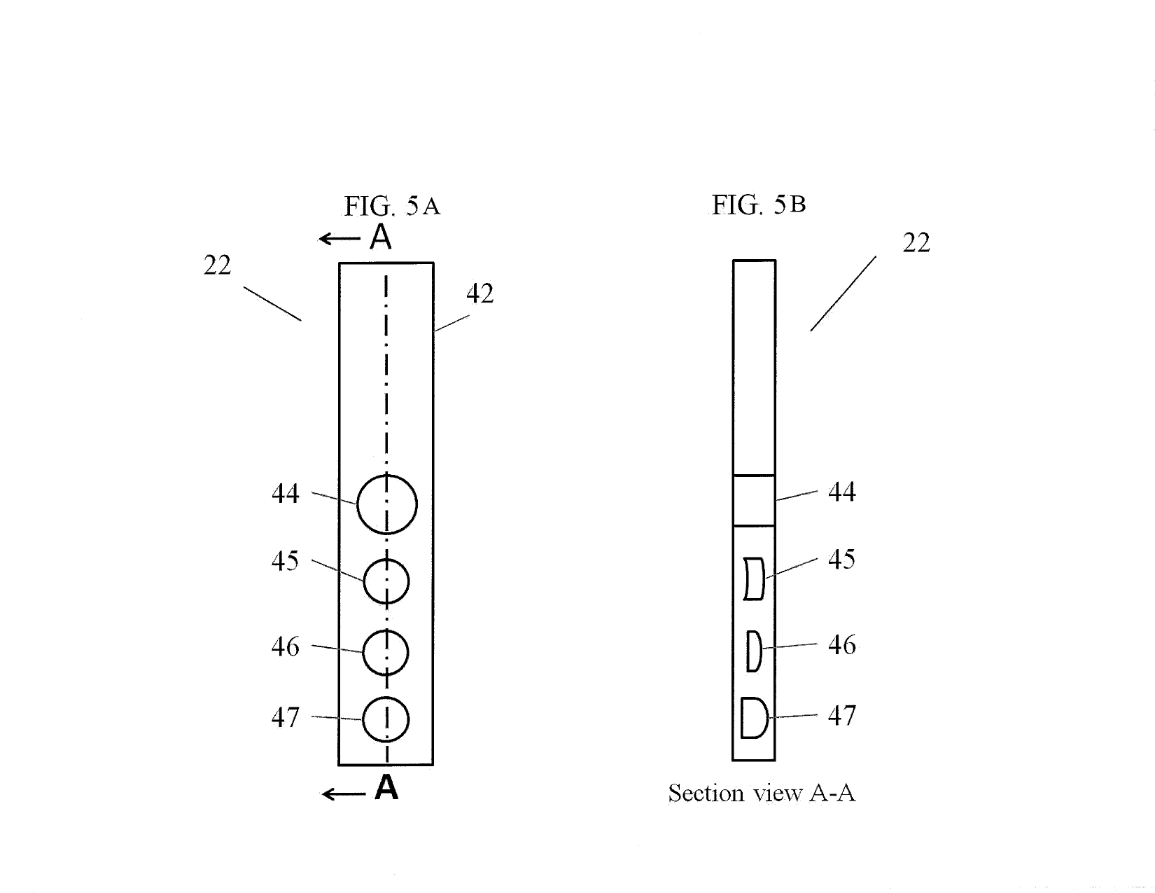

According to some embodiments, a lens selection module, such as the lens slider 22, can be used to achieve adequate focus range for different eye conditions during image acquisition. As shown in FIG. 5A, slider 22 may be a light blocking material 42 with multiple transparent areas. As shown in FIG. 5A, slider 22 includes a first position 44, a second position 45, a third position 46, and a fourth position 47. As shown in FIG. 1, slider can be positioned to allow light to pass through one of positions 44, 45, 46, and 47 to arrive at sensor 5. FIG. 5A is a planar view of slider 22 while FIG. 5B is a cross sectional view along A-A.

Slider 22 can be utilized for fundus imaging of patients with a wide range of refractive error. For example, for patient with minor refractive error, the operator can move lens slider 22 to first position 44, which can be an open hole, as shown in FIGS. 5A and 5B. For patients with severe myopia, the lens slider can be moved to a second position 45 for diopter compensation with a weak negative lens. For patients with severe presbyopia or hyperopia, the slider can be moved to third position 46 with a weak positive lens during image acquisition. The fourth position 47, with a strong positive lens, in the exemplary lens slider 22 can be used for imaging the anterior area of the eye. In some embodiments, the operator can image the anterior area of the eye using the system in FIG. 1 by positioning the whole system from its nominal working distance to a distance around two times the nominal value and adjusting for a proper focus. The number of compensation lenses and the ordering positions of the exemplary slider 22 can vary based on the clinical needs and can be understood by a person of ordinary skill in the art.

To capture a fundus image using the system in FIG. 1, an operator will first align the system to a patient's eye under examination by positioning the system so that lens 1 is about 2″ to 5″ away from the cornea of the eye and adjust the system laterally (X-Y) so the image of the pupil of the patient's eye is centered in the NIR video image on the display device (not shown) that displays the image captured by sensor 5 during the observation mode. In the observation mode, both the focus index illuminating light source (NIR) 10 and the LED ring array (NIR) 19b are turned on to illuminate the focus index and the fundus during the observation mode. Then, the operator can move the system toward the patient's eye until the image of a working distance indicator (not shown), e.g. a dual luminous spots, commonly used in conventional fundus camera, becomes sharp. Now, the image of the fundus and the focus index 9c are shown on the display with the correct working distance. The operator can then instruct the patient to look at one of the fixation target(s) 9d for focusing.

When the two halves of the split-bar pattern of the focusing index are aligned, the imaging mode can be triggered to acquire the fundus image. The imaging mode can be triggered by a commonly used user's input, such as a button press on a joystick control, a mouse click, a foot rest. When the imaging mode is triggered, the focus index optics assembly 9 will flip away quickly from the light path as described above. The focus index illuminating light source 10 and the LED ring array (NIR) 19b will also be turned off and the white LED arrays 19a will then flash the fundus for capturing a fundus image by the image sensor 5.

The above examples are provided in order to demonstrate and further illustrate certain embodiments and aspects of the present invention and are not to be construed as limiting the scope thereof. In the description above, reference is made primarily to the eye as the object. This has to be understood as merely a way to help the description and not as a restriction of the application of the present invention. As such, where the term “eye” is used, a more general transparent and scattering object or organ may be sought instead. Although various embodiments that incorporate the teachings of the present invention have been illustrated and described in detail herein, a person of ordinary skill in the art can readily device other various embodiments that incorporate the teachings of this subject invention.

We claim:

1. An ophthalmic imaging apparatus for capturing images of an eye, comprising:
a fundus illumination system, the fundus illumination system includes a spatially interlaced light source array of one or more wavelength bands and a focus index illumination light source where the focus index illumination light source is mounted on a non-moving part of the ophthalmic imaging apparatus;
a focus index optical assembly; and
a fundus imaging system.

2. The apparatus of claim 1, wherein a light beam from the focus index illumination light is branched into a fundus illumination light path through a folding mirror separated from the focus index optical assembly.

3. The apparatus of claim 2, wherein the folding mirror is placed behind a crystalline lens diaphragm.

4. The apparatus of claim 1, wherein the fundus imaging system includes a sensor capable of detecting one or more wavelength of illumination.

5. The apparatus of claim 1, wherein the light source array includes a plurality of LEDs spaced evenly in a circular configuration.

6. The apparatus of claim 5, wherein the light source array includes sources for two wavelength bands.

7. The apparatus of claim 6, wherein the two wavelength bands include a visible light band and a near infra-red (NIR) light band

8. The apparatus of claim 6, wherein the light source array comprises two layers of LEDs, the first layer including LEDs of the visible wavelength band and the second layer including LEDs of the NIR band.

9. The apparatus of claim 6, wherein the light source array comprises two layers of LEDs, the first layer including LEDs of the NIR band and the second layer including LEDs of the visible wavelength band.

10. The apparatus of claim 6, wherein the light source array comprises two layers of LEDs, the first layer includes a mixture LEDs of the visible wavelength band and the NIR band evenly spaced apart, and the second layer includes a mixture LEDs of the visible wavelength band and the NIR band spatially interlaced with the first layer.

11. The apparatus of claim 1, further comprising a crystalline lens diaphragm with light blocking materials and a prism mirror to direct illumination to the focus index optical assembly.

12. The apparatus of claim 1, wherein the focus index optical assembly comprises a translucent plate, a pattern of light-blocking material, and a bi-prism.

13. The apparatus of claim 12, wherein the pattern of light-blocking material include a focus index and one or more fixation targets.

14. The apparatus of claim 13, wherein the focus index is a slit opening surrounded by a light-blocking central disk.

15. The apparatus of claim 13, wherein the fixation targets are light-blocking areas or small openings where the light-blocking areas or small openings can be of different shapes and sizes.

16. The apparatus of claim 13, wherein the bi-prism is attached on top of the focus index to deflect incident beam into two opposite directions.

17. The apparatus of claim 1, further comprising a diopter compensation assembly where the diopter compensation assembly can be a slider with one opening and one or more diopter compensation lenses.

18. The apparatus of claim 17, wherein the diopter compensation assembly comprises an opening, a negative compensation lens, and a positive compensation lens, the diopter compensation assembly can be configured to be capable of switching between the opening, the negative compensation lens, and the positive compensation lens.

19. The apparatus of claim 1, wherein the focus index assembly is fastened on a solenoid capable of moving in-and-out of the optical path of the apparatus.

20. The apparatus of claim 19, wherein the solenoid is coupled to the sensor capable of movement for focus adjustment.
